(19) United States
(12) Patent Application Publication
(10) Pub. No.: US 2013/0253582 A1
(43) Pub. Date: Sep. 26, 2013

(54) Backrest Apparatus Comprising a Concave Support Pad with Convex End Portions

(71) Applicants: Yako Merogi, (US); Medlum Merogi, (US); Hivi Merogi, (US)

(72) Inventor: Frank TIELVE, North York (CA)

(21) Appl. No.: 13/899,154

(22) Filed: May 21, 2013

Related U.S. Application Data
(63) Continuation of application No. PCT/CA2010/001872, filed on Nov. 24, 2010.

(51) Int. Cl. A61B 17/66

(52) U.S. Cl. CPC A61B 17/66
USPC 606/240

(57) ABSTRACT

A physical therapy apparatus includes an elongated body support sized to substantially support the user's torso with the user's spinal column oriented and aligned in positions which maximize the intervertebral spacing along the anterior regions of the spinal column. The body support is provided with a support pad which extends from a forward end to a rearward end having a longitudinal length of between about 0.6 and 2 meters. An upwardly curving convex portion is positioned adjacent each of the forward and rearward ends, with an upwardly middle concave saddle portion connecting each convex portions. A first convex portion has a curvature selected to provide supporting contact to the user's lumbar and thoracic regions of the spine in the performance of one procedure, and the sacral and pelvic regions in another procedure. Optionally, the concave saddle portion may be scalloped or otherwise contoured to facilitate the proper alignment of the user's buttocks and legs in one procedure and the upper back and shoulders in another procedure thereon.

### CROSS REFERENCE TO RELATED APPLICATIONS

This application is a Continuation of PCT Application number PCT/CA2010/001872, filed on Nov. 24, 2010. The aforementioned related application is incorporated herein by reference in its entirety.

### FIELD OF THE INVENTION

The present invention relates to a physical therapy apparatus, and more particularly a physical therapy apparatus which is suited for the self-administered alignment of spinal vertebrae, re-centering of spinal disc cores, spinal disc and other body joints decompression, and relaxation of soft and connective tissues throughout the body.

### BACKGROUND OF THE INVENTION

The spinal column is a critical part of the skeletal system. It is a flexuous column, formed of a series of vertebrae and associated spinal discs, the vertebrae are divided into true and false. True vertebrae are twenty-four in number, and together form three specific spinal curvature regions: the cervical; thoracic; and lumbar. Nine false vertebrae compose the sacrum and coccyx sections of the spine. The false vertebrae are firmly united and form two bones at the base of the spine. As shown best in FIG. 1, each vertebrae 2 includes a body 4 as its largest and most solid part and together with the spinal discs serve as a primary support for the weight of the cranium and trunk. At the upper and lower regions the vertebral body 4 is slightly concave, presenting a rim around its circumference, with roughened surfaces for the attachment of intervertebral discs. Among other features, the vertebrae 2 contain three different types of processes: a Spinous Process 6, Transverse Process 8a,8b and Articular Process 10. The Spinous Process 6 projects posteriorly from the junction of the two laminae, and serves as an attachment point for muscles. The Transverse Process 8a,8b, project from each side of the body 4 at the point where the Articular Process 10 joins the Pedicle 12, and serve as attachment surfaces for muscles. The Articular Process 10 further includes two superior articular processes and two inferior articular processes.

The vertebrae and intervertebral discs vary in size depending on their position along the spinal column, with the largest being in the lumbar region and the smallest in the cervical region. At the center of the discs the nucleus is of a substance that possesses liquid properties and resists compressive forces. A series of annular rings of limited flexibility that reinforce each other maintain the nucleus in the center of the discs, with nerve sensors found along the outer rings. Because of the Processes 6,8a,8b,10, the spine has a greater range of motion when flexing forward than when extending backwards. As a result, the pressure exerted on the annular rings about the disc nucleuses is greater when the spine is fully flexed anteriorly, than when fully extended posteriorly. Excessive forward flexion of the spinal column due to improper posture or body motion tends to cause undue stress to the posterior side of the annular rings. In time, this will cause disc damage by the gradual over-stretching and tearing of inner annular rings, and eventually the over-stretching and tearing of outer annular rings. When the inner annular rings are over-stretched and torn, the outer annular rings will in turn also begin to over-stretch. Overstretched outer annular rings may eventually form a bulge or herniation on the posterior side of the spinal discs. If all the outer annular rings in a particular disc over-stretch and tear, the affected disc may extrude the nucleus into the spinal canal.

Disc herniation can apply undue pressure to nearby nerve bundles and/or to the spinal cord, potentially causing neurological dysfunctions that may, directly or indirectly affect any part of the body. Because the spinal column is of great importance, the brain will in the case of undue spinal disc stress or damage trigger protective muscle and other soft tissue contraction to limit or prevent the motion causing the disc stress or damage. Because segmental muscle fibers that connect the vertebrae to each other attach to several vertebrae, the protective segmental muscle contraction tends to gradually adversely affect other nearby spinal discs by means of chronic compression, potentially causing a far reaching chain reaction of contracting soft tissues.

In addition, undue chronic compression of spinal discs prevents their proper hydration which is essential for their good health. In extreme cases of insufficient disc hydration, the discs may become thin enough that adjacent vertebrae may effectively fuse. Thinning spinal discs are a common condition presently erroneously labeled as degenerative disc disease.

Contraction of soft tissues throughout the body triggered by spinal disc damage will cause the compression of body joints and vital systems. Compressed vital systems will have increasingly reduced operating efficiencies, leading to the development of an extensive number of symptom ailments, the reduction of health, quality of life and lifespan.

### SUMMARY OF THE INVENTION

When a person maintains proper posture (the full natural curves of the spine), the disc spaces between the vertebrae are either of equal height on the anterior and posterior sides of the spinal column, or have a greater height on the anterior side. With proper posture, downward pressure exerted on the spinal column will cause the disc cores to either stay centered when the vertebral surfaces are essentially parallel to each other, or exert a moderate non-damaging force towards the anterior side of the disc's annular rings. When the natural spinal curvatures of the lumbar and cervical sections of the spine are reduced, and the thoracic curvature is increased (which occur with improper posture), a wedge shape space is formed between the vertebrae with the greater height on the posterior side of the disc spaces, causing the disc cores to apply undue, damaging pressure to the posterior side of the spinal disc's annular rings.

The present invention seeks to provide an apparatus which is configured to facilitate spinal alignment and/or disc decompression, as for example which may be advantageously used to offset the compressive effects on the spinal discs associated with external physiological conditions such as improper posture habits, or trauma.

Another object of the invention is to provide a physical therapy apparatus which may be used to increase hydration of spinal discs, that is essential to maintain healthy discs, or for the healing of damaged discs.

Another object of the invention is to provide a simplified apparatus that uses the force of gravity and leverage to effect stretching forces necessary to achieve an enhanced soft tissue manipulation of a user's spine, and the re-centering of disc cores.

A further object of the invention is to provide a physical therapy apparatus for the simplified decompression and/or alignment of a user's vertebral column, which is configured to allow a user to self-manipulate his or her vertebrae, spinal discs and soft tissues in an optimum orientation.

A further object of the present invention is to provide, an inexpensive physical therapy apparatus which is configured to provide physical therapy to a user by stretching powerful ligaments that surround the spinal column which contracted on the anterior side, over time, primarily because of improper posture, increasing the posterior spacing between the vertebrae. More preferably, the apparatus is configured to support the user thereon with the spine positioned with optimum curvatures to advantageously manipulate the spinal vertebrae, spinal discs and other related soft and connective tissues.

Another object of the invention is to provide a physical therapy apparatus that effectively decompresses body joints and relaxes the overall muscular system. Relaxation of the muscular system decompresses other vital systems, leading to their greater operation efficiency and improved health. The relaxed muscular system can then be developed to its full potential.

In one possible embodiment, the present invention provides a physical therapy apparatus which is adapted for either self-administered or third party professional administered therapy. The apparatus is configured to effect the stretching of soft tissues associated with the spinal column, and indirectly, soft tissues throughout the body. Also, the decompression of the anterior regions of the vertebral discs, therefore facilitating disc core re-centering, disc hydration and healing. In a simplified construction, the physical therapy apparatus includes an elongated body support which is sized to substantially support the user's torso thereon. The body support has a shape configured to position the torso in an orientation such that the vertebrae are generally longitudinally aligned along the length of the spinal column, and the user's back is arched to provide amongst other benefits, increased anterior disc spacing, anterior disc core displacement and enhanced disc hydration. Preferably, the body support is provided with a user supporting sling, board, panel, or other suitable supporting pad (hereinafter collectively referred to as a support pad) which is configured to support the user with the shoulders and hips aligned in reclined positions.

The support pad extends longitudinally along an axis from a forward end to a rearward end, having a longitudinal length which is selected to be at least as long as an average individual's torso, and preferably extends in length between 0.6 and 2 meters. The support pad is provided with a generally upwardly curving or convex portion positioned adjacent each respective forward and rearward end. An upwardly concave intermediate or middle saddle portion joins each convex portion and assists in maintaining the user in a correct position on the apparatus.

The apparatus is used to perform two basic procedures as illustrated in FIGS. 7 and 8. In a basic construction a first one of the convex end portions is formed having a curvature which is selected to provide supporting contact to the user's thoracic and upper lumbar regions when the user's buttocks are cradled by the concave mid-portion and the user reclines rearwardly against the support pad as illustrated in FIG. 7, so that the posterior side of the spinal column is in juxtaposed contact therewith. The curvature of the first convex portion is chosen so that by positioning the thoracic and lumbar spine sections against the upwardly convex surface, the user's vertebrae assume an orientation wherein the intervertebral disc spaces along the anterior side of the spinal column are increased relative to the spacing along the posterior side. The applicant has appreciated that the said disc spacing change advantageously decompresses the discs, contributes to the re-centering of disc cores, and effectively stretches powerful ligaments on the anterior side of the spine, therefore, facilitates disc hydration and healing.

In the procedure illustrated in FIG. 8, the user's sacral and pelvic sections are supported by the first convex portion, while the upper back and shoulders are cradled by part of the concave mid-portion and the neck is cradled by a central concave portion of the second convex form. The applicant has appreciated that this procedure provides the forces required to positively affect the structures associated with the lower lumbar region which are particularly difficult to manipulate.

Although not essential, in a more preferred construction, the concave saddle portion may be scalloped or contoured to facilitate seating and the alignment of the user's buttocks and upper legs thereon when performing the procedure illustrated in FIG. 7, or the upper back and shoulders when performing the procedure illustrated in FIG. 8.

It is furthermore envisioned that in an alternate embodiment the support pad could be adjustable in length. For example, the support pad may be provided with one or more foldable, stretchable, or removable segments which allow for the longitudinal extension or retraction of the support pad to facilitate optimum use by a number of users of differing heights or weights.

Accordingly, one aspect of the present invention resides in a physical therapy apparatus for effecting spinal disc decompression, disc core re-centering, alignment of the user's vertebrae, decompression of body joints and relaxation of soft tissues throughout the body, the apparatus including: a frame and a support pad for providing support contact to the user's spinal column when the user is in reclined positions thereon, the support pad being longitudinally elongated along a central axis from a first end portion to a second end portion, in a longitudinally side profile each of the first and second end portions extending convexly upwardly and being separated by an upwardly concave intermediate mid-portion.

Another aspect of the present invention resides in a physical therapy apparatus for effecting disc decompression, disc core re-centering, alignment of the user's vertebrae, decompression of joints and relaxation of soft tissues throughout the body, the apparatus including: a frame and a support pad for providing supporting contact to the user's spinal column when the user is in a position reclined thereon, the support pad being longitudinally elongated along a central axis from a first end portion to a second end portion, in a longitudinal side profile each of the first and second end portions extending convexly upwardly and being separated by an upwardly concave intermediate mid-portion, the first end portion having a curvature selected to support the user's thoracic and upper lumbar sections of the spine in juxtaposed contact therewith when performing the procedure illustrated in FIG. 7, with an increased spacing at the anterior side of the user's upper lumbar, thoracic and cervical vertebrae relative to the posterior side. The first convex portion including an inclined surface for supporting the user's upper lumbar and thoracic vertebrae when the user's spine is positioned in juxtaposed contact therewith, the inclined surface extending at an angle selected at about 30° to 50°±10° relative to horizontal.

Another aspect of the present invention resides in a physical therapy apparatus for effecting disc decompression, disc core re-centering, alignment of the user's vertebrae, decompression of body joints and relaxation of soft tissues throughout the body, the apparatus including: a frame and support pad for providing supporting contact to the user's sacral and pelvic structures when the user is in a position reclined thereon, the support pad being longitudinally elongated along a central axis from a first end portion to a second end portion, in a longitudinally side profile, each of the first and second end portions extending convexly upwardly and being separated by an upwardly concave intermediate mid-portion, the first convex end portion having a curvature selected to support the user's sacral section and pelvic structure in juxtaposed contact therewith when performing the procedure illustrated in FIG. 8, with an increased spacing at the anterior side of the user's lumbar and thoracic vertebrae relative to the posterior side.

A further aspect of the present invention resides in a physical therapy apparatus for effecting decompression of the user's spinal discs, the apparatus including: a frame and a body supporting pad mounted on said frame, the pad having a first generally upwardly convex portion and a generally upwardly second convex portion, the body supporting pad extending longitudinally along an axis and including: an upwardly concave portion intermediate to and connecting said first and second convex portions, the first convex portion including a generally axially disposed raised member and a pair of recessed portions, each recessed portion disposed on each lateral side of the raised member sized to accommodate the protruding shoulder blades when the user is performing the procedure illustrated in FIG. 7, or the pelvic structure when the user is performing the procedure illustrated in FIG. 8.

A further aspect of the present invention resides in a physical therapy apparatus for effecting decompression of the user's spinal discs, the apparatus including: a frame and a body supporting pad mounted on said frame extending from a first generally upwardly convex portion to a second generally upwardly convex portion, the body supporting pad extending longitudinally along an axis and including: an upwardly concave portion intermediate to and connecting said first and second convex portions, the second convex portion including a central recessed portion sized to accommodate the user's cervical section in an essentially neutral position when the user performs the procedure illustrated in FIG. 8. Adjacent to the said recessed cervical section supporting feature there are two additional axially aligned recessed portions designed to support the user's legs in an optimum angle of orientation in order to maximize the efficiency of the procedure illustrated in FIG. 7.

### BRIEF DESCRIPTION OF THE DRAWINGS

Reference may now be had to the following detailed description taken together with accompany drawings, in which:

FIG. 1 shows a schematic view illustrating the general structure of a human vertebra;

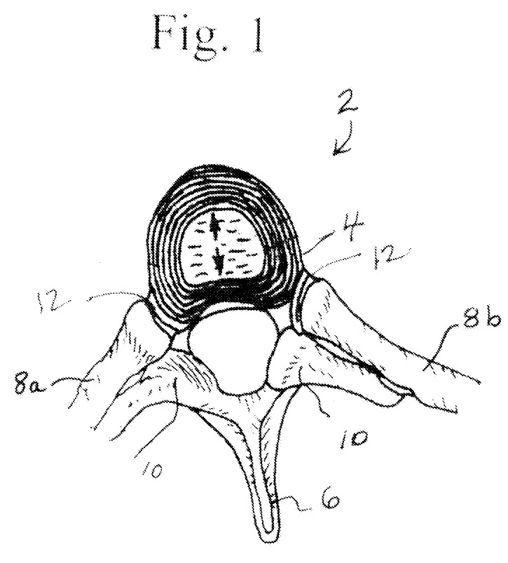

FIG. 2 shows a schematic side view of a physical therapy apparatus in accordance with a preferred embodiment of the invention;

FIG. 3 shows a schematic view of the forward end of the physical therapy apparatus shown in FIG. 2;

FIG. 4 shows a schematic view of the rearward end of the physical therapy apparatus shown in FIG. 2;

FIG. 5 shows a schematic top side view of the physical therapy apparatus shown in FIG. 2;

FIG. 6 shows a cross-sectional view of the support pad used in the physical therapy apparatus shown in FIG. 5 taken along 6-6′;

FIG. 7 shows a schematic side view of a user in a first forwardly reclined position on the physical therapy apparatus of FIG. 2, with the posterior side of the spinal column interacting with the support pad in a first spinal alignment and stretching position;

FIG. 8 shows a schematic side view of a user in a second rearwardly inclined position, on the physical therapy apparatus of FIG. 2, with the sacral, pelvic, upper back, shoulders and cervical sections interacting with the support pad in a second therapeutic position;

FIG. 9 shows a perspective view of a foot weight for use with the apparatus of FIG. 2.

### DESCRIPTION OF THE INVENTION

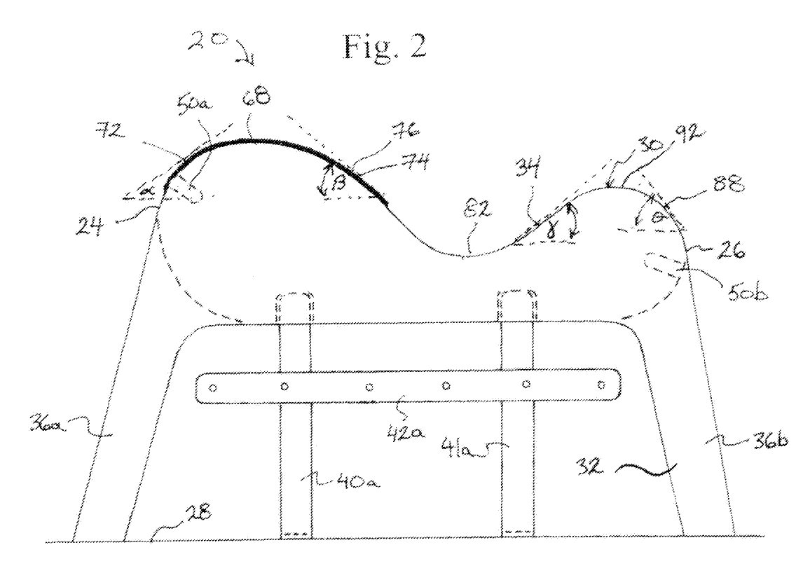

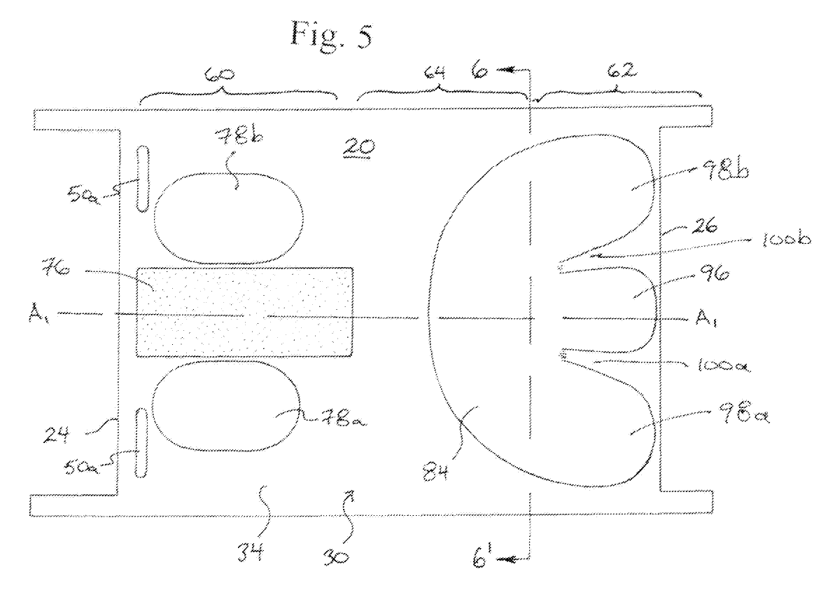

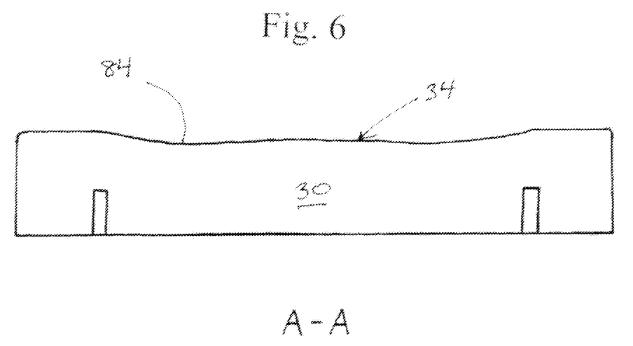

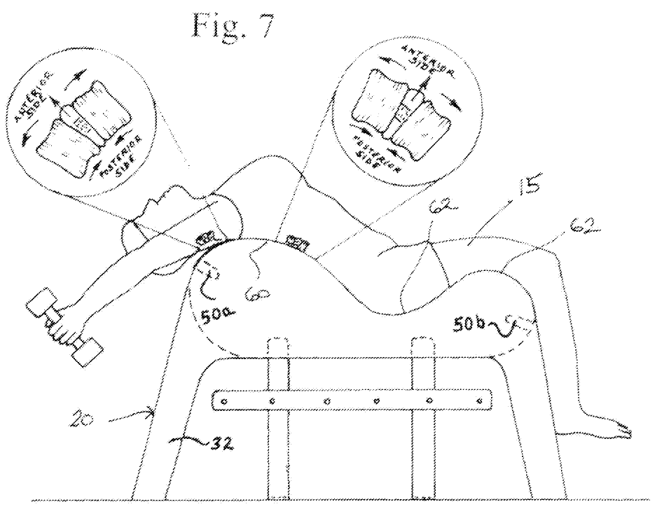

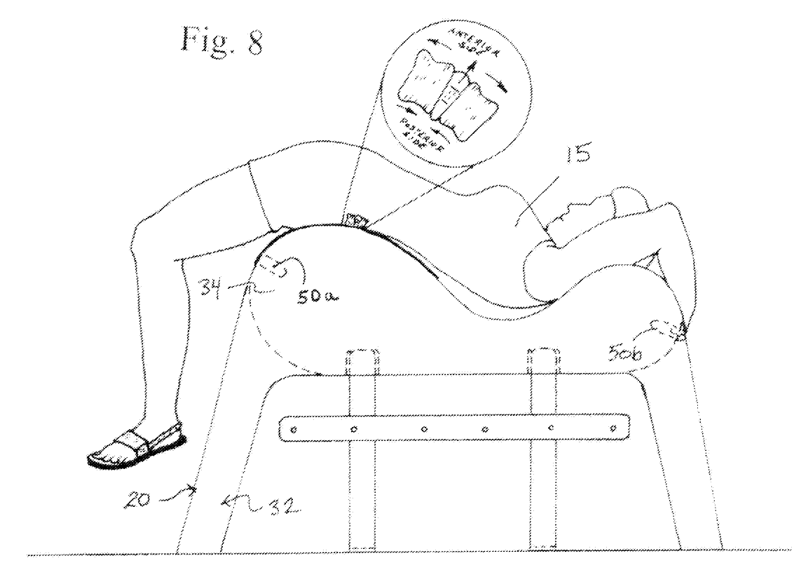

Reference is made to FIGS. 2 to 6 which show a floor supported physical therapy apparatus 20 used in the self-administered manipulation by a user 15 (FIGS. 7 and 8) of the user's spine and major body joints, including the spinal discs in accordance with a preferred embodiment of the invention. The apparatus 20 is elongated along a longitudinal central axis Aᵣ A₁, (FIG. 5) extending from a forward end 24 to a rearward end 26. The apparatus 20 includes a body support 30 and a frame assembly 32 which includes two pairs of support legs 36a,36b. As will be described hereafter, the body support 30 is secured to the frame assembly 32 in a position spaced approximately 0.4 to 0.7 meters above the floor 28.

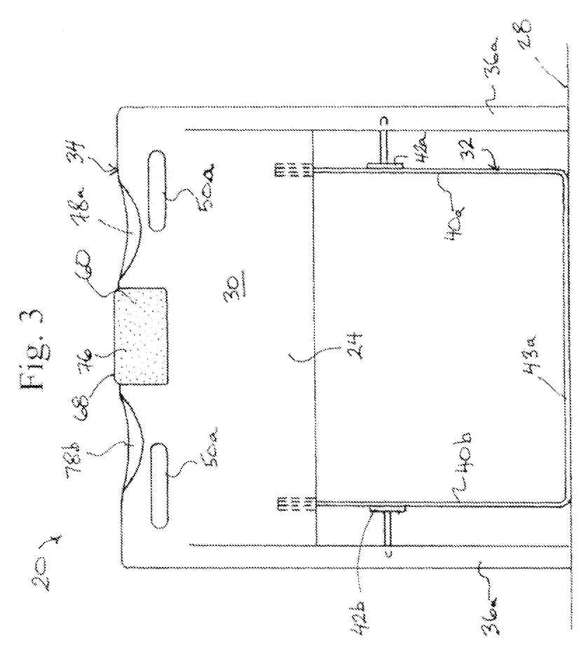

As shown best in FIGS. 2 and 3, the body support 30 includes a body support pad 34 supported at each of its ends by the support legs 36a,36b. The support pad 34 has an overall longitudinal length and width chosen to comfortably support the user 15 in upwardly facing reclined positions thereon. Preferably, the support pad 34 is generally elongated longitudinally in the direction of the axis A₁-A₁, having an overall longitudinal length selected at between 1 and 2 meters, and preferably about 1.5 meters. In a lateral direction, the support pad 34 is symmetrically formed about the axis A₁-A₁, and has a lateral width selected at between about 0.5 and 0.8 meters. The body support 34 is preferably formed of injection molded plastic having an overall rigid or substantially semi-rigid construction. Preferably, the body support pad 34 and supporting legs 36a,36b are injection molded as a single unit.

The support pad 34 is constructed to comfortably support the user 15 thereon, while maintaining the user's hips, shoulders and legs alignment with each other, and the torso in a position centered with the axis A₁-A₁. As will be described, the curvature of the pad 34 is selected to ensure the regions of the spine orient longitudinally with an optimal curvature relative to each other to maximize disc core re-centering, disc decompression and hydration.

If necessary, the pad 34 may be provided with internal reinforcement (not shown). The support legs 36a,36b are preferably located along a respective longitudinal side of the support pad 34 to provide both enhanced stability and possible anchoring structures for any internal reinforcement.

The frame assembly 32 further includes secondary supports 40a,40b and 41a,41b which couple to the underside of the body support pad 34 for added strength and stability. Optionally, two pairs of gripping handles or recesses 50a,50b may be molded into the support pad 34 towards each of its axial ends 24,26, respectively. The gripping recesses 50a,50b are sized and positioned for easy gripping by the user's hands to both facilitate the mounting and dismounting onto and from the body support pad 34, and to maintain the user's torso in the desired positioning on the support pad 34 during the performance of the procedures illustrated in FIGS. 7 and 8.

As shown in FIGS. 2 and 3, the forward and rearward pairs of vertical supports 40a,40b and 41a,41b are fixed in position by lateral cross-braced members 42a,42b. Preferably, the pairs of vertical supports 40a,40b extend upwardly away from the other to engagingly support the underside of the support pad 34. Optionally, the vertical supports extend at an angle of between about 65° and 90° from horizontal, providing me physical therapy apparatus 20 with both increased stability and load capacity. Alternatively, the described vertical supports 40a,40b and 41a,41b may be injection molded in plastic together with the lateral cross-braced members 42a, 42b as a single unit.

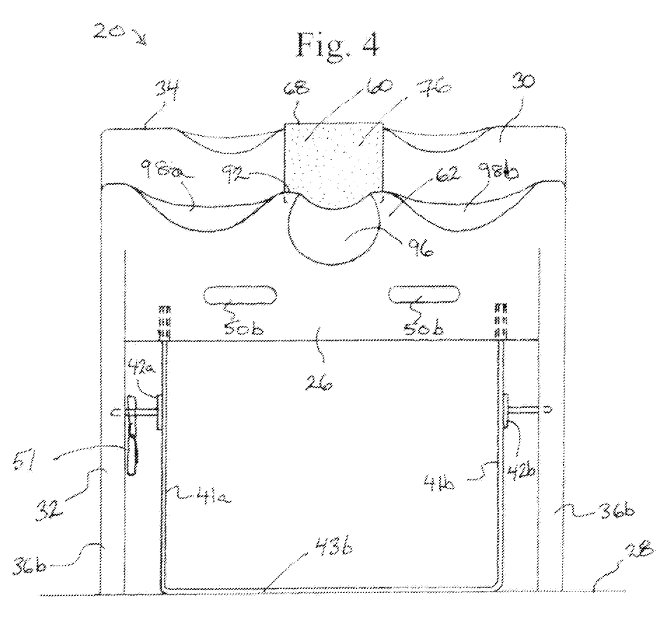

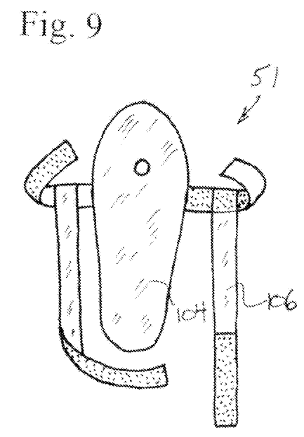

FIGS. 3 and 4 show best the positioning of the body support pad 30 on the frame assembly 32. In a simplified construction, a pair of lower cross-brace members 42a,42b are secured respectively to each of the vertical supports 40a,41a and 40b,41b along the floor 28. In addition to providing enhanced structural rigidity to the frame assembly 32, the cross-braces 42a,42b function as a mounting rack for foot weights 51 (FIG. 9) and hand-held weights used to increase the efficiency of the procedures illustrated in FIGS. 7 and 8.

As will be described, the foot weights 51 and hand-held weights are selected to provide increased resistive and tensional forces in the self-administered manipulation of the user's spine and limbs.

The support pad 34 is provided with an overall shape and contour selected to orient the user's torso, neck and hips in a general alignment centered along with the axis A₁-A₁, with the spinal column positioned with an optimum alignment and curvature to effect the stretching and relaxation of vertebral associated tissues, while effecting the decompression of the anterior regions of the user's vertebral discs therefore, aiding the re-centering of disc cores and increasing disc hydration. Preferably, the support pad 34 has curvatures selected to orient one or more regions of the user's spine so that the vertebrae spacing along the anterior side of the spinal column is increased relative to that of the posterior side. Reference is had to FIGS. 2 to 4 which illustrate best the body support pad 34 used in the physical therapy apparatus 20 in accordance with a preferred embodiment of the invention. FIG. 2, shows a longitudinal side profile of the support pad 34 as including a major convexly upwardly curving forward portion 60 which is located adjacent to the forward end 24 of the apparatus 20; and a minor convexly upwardly curving rearward portion 62 which is located adjacent to the rearward end 26. An upwardly concave central saddle portion 64 connects and merges with the forward and rearward portions 60,62.

As shown in FIG. 7, in side profile, the major convex forward portion 60 is provided with a curvature which is selected to interact with the user's upper lumbar and thoracic vertebrae when the user 15 reclines in the forward position shown with the posterior side of the spinal column pressing against the body support pad 34. The curvature of the forward portion 60 is chosen such that juxtaposed contact occurs primarily through the upper lumbar and thoracic sections of the spinal column and the user's head is freely suspended with its weight effecting therapeutic curvature in the cervical section of the spine.

In the position shown, the convex forward portion 60 reorients upper lumbar, thoracic and cervical vertebrae with an increased anterior spacing along the anterior side of the spinal column, relative to the posterior side. More preferably, the curvature of the convex portion is chosen to provide maximum intervertebral disc spacing along the anterior side of the upper lumbar, thoracic and cervical vertebrae. Although the forward portion 60 could be formed having a circular curvature, most preferably, the portion 60 is formed having an irregular or partially elliptical profile. FIG. 2 shows the convex forward portion 60 extending longitudinally with a forward most surface 72 extending upwardly at an angle a of between about 55° and 75°±10°, and preferably about 50° ±10 apex. From the apex 68, the convex portion 60 curves downwardly along a back surface 74 at an angle β of between about 25° and 45°±10 relative to horizontal and preferably at an angle β of between about 30° and 40°±10.

As shown best in FIG. 5 along its lateral extent, the convex forward portion 60 is symmetrical about the axis A₁-A₁. An axially positioned raised elastomeric support 76 is provided along the curvature of the forward portion 60. The elastomeric support 76 is preferably in the form of a resiliently compressible elastomeric cushion which in use supports the thoracic section of the spine when performing the procedure illustrated in FIG. 7 with the user's cervical section and head hanging freely, and supports the sacral section of the spine when the user performs the procedure illustrated in FIG. 8. Spaced on each lateral side of the elastomeric support 76 are a pair of upper recesses 78a,78b. Each of the recesses 78a,78b extend longitudinally across the apex 68, and partway into the upward and downwardly sloping surfaces 72,74. As will be described, the upper recesses 78a,78b have a lateral width which is chosen to cradle a respective one of the user's pelvic bones and shoulder blades therein, where by when the user reclines rearwardly on the apparatus 20, the recesses 78a,78b assist in the positioning and alignment of the user's torso on the support pad 34 and prevent excessive pressure on protruding pelvic structures while performing the procedure illustrated in FIG. 8. Similarly, the recesses 78a,78b assist in the positioning and alignment of the user's torso while relieving excessive pressure on protruding shoulder blades while the user performs the procedure illustrated in FIG. 7.

The forward convex portion 60 merges rearwardly into the concave central saddle portion 64. The concave central portion 64 has a curvature selected to provide supporting contact with the user's lumbar and sacral vertebrae when the user 15 reclines forwardly with the posterior side of his spinal column resting against the support pad 34. Along the longitudinal length of central portion 64, the curvature of the central support pad 34 flattens to a lowermost bight 82. Rearwardly from the bight 82, the central portion 64 curves again upwardly at an angle Y of about 30° to 55°±10°, and more preferably about 45°±10° to merge with the convexly upward curvature portion 62. In its lateral extent, the bight 82 is provided with a scalloped or contoured seat 84 (FIG. 5). The seat 84 is shaped and sized to cradle the user's buttocks while preventing axial body sliding movement and assisting in optimum alignment of the spinal column, centered, reclined against the body support 30.

In longitudinal profile, the rearward convex portion 62 is preferably provided with a generally mirror profile of a reduced dimension to that of the forward portion 60. From the rearward end 26 of the apparatus 20, the rear convex portion 62 extends from a rearwardmost surface 88 forwardly and upwardly at an angle θ of between 45° and 75°±10°, and more preferably about 40 to 55°±10° from horizontal, to an uppermost rear apex 92. In a most preferred construction, the apex 68 of the forward portion 60 locates between about 0.1 to 0.4 meters vertically above the apex 92 of the rearward convex portion 62. Forwardly of the apex 92, the convex portion 62 slopes downwardly, merging into the concave central saddle portion 64. FIGS. 4 and 5 show the rearward convex portion 62 as further provided in lateral cross section, with an axially aligned central recess 96, and a pair of side lower recesses 98a,98b. Each of the lower recesses 98a,98b are symmetrically spaced on a respective lateral side of the central recess 96. The recesses 96 and 98a,98b define respectively a pair of longitudinally extending ridges 100a, 100b therebetween symmetrically disposed along each lateral side of the axis Aₗ-A₁.

The recesses 96 and 98a,98b extend substantially the longitudinal length of the convex rearward portion 62, with the side recesses 98a,98b tapering outwardly away from the axis Ax-Ai in a rearward direction, to assist in positioning the user's legs in a general outwardly spreading orientation, with his or her knees generally in alignment in a width-wise direction with the user's shoulders.

The rearward convex portion 62 is provided with an overall curvature selected to support the underside of the user's upper legs when the user 15 reclines with the spine in juxtaposed contact with the body support 30 with the thoracic vertebrae resting against the support 76. More preferably, however the curvature of the rear portion 62 is selected to further support the upper portion of the user's back and cervical section of the spine when the user 15 reclines rearwardly with the posterior side of the spinal column resting against the support pad 34, and user's cervical section 15 cradled within the central recess 96 with the user's head oriented towards the rearmost end 26 of the apparatus. In this configuration, the inward taper of the side recesses 98a, 98b advantageously allow for cradling of the user's shoulder blades for enhanced stability, comfort and spinal alignment.

FIGS. 7 and 8 illustrate the preferred use of the physical therapy apparatus 20 in a self-administered therapy for the spinal alignment, and decompression of the user's spinal discs. As shown best in FIG. 7, in an initial use, the user 15 assumes a forward position with the upper lumbar and thoracic vertebrae reclined against the support pad 34 and with the head suspended and positioned forwardly therepast. In the orientation shown, the user's buttocks are positioned against the contoured bight 84 and the cervical region of the spinal column is suspended above the support 30, allowing the cervical region increased curvature by the weight of the head. Each of the user's legs is further extended with a respective upper leg resting in a lower recess 98a, 98b, with the user's lower legs draped over the rearward convex portion 62 so as to hang downwardly therefrom. The user 15 fully reclines against the support pad 34 arching the back so that only the upper lumbar and thoracic vertebrae are moved into engaging contact against the forward convex portion 60 and the user's head and cervical vertebrae are suspended forwardly therepast. In the position shown, the thoracic region interacts with the elastomeric pad 76 for increased comfort. As shown, with the user's spine resting against the convex portion 60, the vertebrae in the upper lumbar, thoracic and cervical sections of the spine are moved so that the disc spaces along the anterior side of the spine are substantially greater than the disc spaces on the posterior side. The position and realignment of the spine results in the re-centering of disc cores, decompression of the discs and improved disc hydration. Optionally, to provide increased efficiency, the user may grasp hand weights when performing the procedure illustrated in FIG. 7, or foot weights when performing the procedure illustrated in FIG. 8. In a simplified construction, the foot weights 51 which are shown in FIG. 9 are provided as metal plates with a variety of weights to match the user's capacity. The metal plates 104 are adapted to preferably be strapped to the user's feet by means of Velcro™ fastening strips 106, although other attachment options can be easily envisioned. In addition to the said benefits, using the handheld and feet weights facilitate the relaxation of muscles in the limbs, the decompression of limb joints, and muscle development.

In a second mode of operation shown in FIG. 8, stretching of the spinal column and decompression of the spinal discs of the lumbar and lower thoracic regions is effected with the user in a reversed reclined position on the apparatus 20. In the reversed reclined position, the user 15 rests against the support pad 34, with the back of the neck cradled in the central recess 96. Here, the user's cervical section of the spine is supported in a generally neutral position with approximately equal disc space on the anterior and posterior sides of the vertebrae. In the position shown, the user's shoulder blades and shoulders further fit into a respective side lower recess 98a,98b to restrict any rearward sliding movement along the support pad, and increase comfort. The user's pelvic bones adjacent to the sacral plate are cradled within a respective upper recess 78a,78b to ensure alignment of the user's shoulders and hips, as well as that of the user's torso with the axis A₁-A₁, reducing pressure on the pelvic structure and increasing comfort.

In the position shown in FIG. 8, contact with the support pad 34 is primarily concentrated in the sacral and pelvic areas, as well as the user's shoulders. This increases spinal column curvature along the lumbar and thoracic regions, with increased disc space height along the anterior side of the lumbar and thoracic regions, relative to the posterior side. To effect spinal decompression, the user 15, gripping the handle recesses 50b, arches the back while extending the legs outwardly past the front end of the support pad 34 to effect maximum curvature of the lower regions of the spine.

The use of foot weights 51 advantageously allows for still increased leveraging forces on the spinal column, to provide enhanced de-compressive force of the lower back region, as well as decompression of the hip, knee and ankle joints.

Although the preferred embodiment describes a unitary and rigid, or semi-rigid support pad 34 mounted on a fixed frame assembly 32, the invention is not so limited. In an alternate embodiment the physical therapy apparatus could include a support pad having multiple components or attachments to effect curvature changes to forward and rearward portions 60,62 for different user's of varying height. Also, a variable frequency vibrator may be attached to a separate plate under the elastomeric pad section 76 which would be isolated from the rest of the apparatus 20 by means of flexible vibrator mounts (not shown).

While the preferred embodiment of the invention describes the body support pad 34 as being of a rigid, or semi-rigid construction made of injection molded plastic resin, the invention is not so limited. It is to be appreciated that in an alternate construction, the support pad 34 could be formed from substantially incompressible fiberglass resin, wood, metal or other materials. Alternatively, the support pad 34 could be provided with multiple resiliently compressible cushion portions, as for example, across the concave central portion 64 for increased comfort.

Although the detailed description of the preferred embodiment describes the use of the physical therapy apparatus 20 by the user 15 in self-administered procedures as previously described, the invention is not so limited. It is to be appreciated that the apparatus 20 is equally suited for third party assistance by for example, professional physiotherapists or chiropractors to effect a patient's spinal alignment and disc decompression. The apparatus 20 furthermore could be incorporated into part of an apparatus used to apply a medical traction force, or a weight or resistance training, the apparatus being adapted to provide stretching and/or decompression forces along the direction of axis A₁-A₁ to better facilitate alignment of the spine, decompression of spinal discs and stretching of spinal related soft tissues.

While the frame assembly 32 is shown with support legs 36a,36b, other supporting constructions are also possible, including without restriction, the use of fixed and/or hingely moveable foot straps, stirrups, pedals, foot pads, and the like.

Although the detailed description describes and illustrates various preferred embodiments, the invention is not so limited. Many modifications and variations will now occur to persons skilled in the art. For a definition of the invention, reference may be had to the appended claims.

1. A physical therapy apparatus for effecting vertebral column alignment, re-centering spinal disc cores, decompression of spinal discs and relaxation of soft tissues, the apparatus including:
a frame and a support pad for providing supporting contact to the user's spinal column when the user is in positions reclined thereon,
the support pad being mounted to a frame and extending along a longitudinal axis from a first end portion to a second end portion, in a longitudinal side profile each of the first and second end portions extending convexly upwardly and being separated by an upwardly concave intermediate mid-portion.

2. The physical therapy apparatus as claimed in claim 1, wherein the first end portion extends in a lateral direction from a first support pad side to a second support pad side, the first end portion including raised lateral side portions, each adjacent the first and second support pad sides, respectively, and
a raised central portion being generally aligned with the axis.

3. The physical therapy apparatus as claimed in claim 2, wherein the raised central portion comprises a resiliently compressible cushion.

4. The physical therapy apparatus as claimed in claim 1, wherein the second end portion extends in a lateral direction from the first support pad side to the second support pad side, the second end portion including a recessed central portion generally aligned with the axis, and a pair of longitudinally elongated lower recessed portions, each lower recessed portions being located intermediate the recessed central portion and a respective one of the first support pad side and the second support pad side.

5. The physical therapy apparatus as claimed in claim 1, wherein the support pad is positioned on the frame a distance of between about 0.3 and 1 meter above the floor, and wherein the support pad has a longitudinal length selected at between about 1 and 2 meters.

6. The physical therapy apparatus as claimed in claim 1, wherein in side profile, the first end portion has a curvature selected to interact with the pelvic section, and the sacral, lumbar and thoracic regions of the spine so that a significantly greater disc space height is achieved on the anterior side of the said sections of the spine relative to the posterior side of the spine.

7. The physical therapy apparatus as claimed in claim 1, wherein in side profile, the second end portion has a curvature selected to support the user's neck and upper back in substantially juxtaposed contact therewith, with the first end portion supporting the sacral and pelvic regions effecting a substantially increased disc space height on the anterior side of the lumbar and thoracic regions of the spine relative to the posterior side.

8. The physical therapy apparatus as claimed in claim 1, wherein the support pad is symmetrical about the longitudinal axis.

9. The physical therapy apparatus as claimed in claim 1, wherein in side profile, an apex of the first end portion locates vertically a distance of between about 0.1 and 0.4 meters above an apex of the second end portion.

10. The physical therapy apparatus as claimed in claim 1, wherein the first and second end portions are upwardly convex and each portion includes a pair of recess handles sized for gripping by the user, each recess disposed symmetrically about the longitudinal axis.

11. The physical therapy apparatus as claimed in claim 1, wherein said support pad includes a semi-rigid compressible surface section.

12. The physical therapy apparatus as claimed in claim 1, wherein said support pad includes at least four handle recesses that are integrally molded.

13. A physical therapy apparatus for effecting vertebral column alignment, re-centering spinal disc cores, decompression of spinal discs and relaxation of soft tissues, the apparatus including:
a frame and a support pad for providing supporting contact to the user's spinal column when the user is in a position reclined thereon, the support pad being longitudinally elongated along a central axis from a first end portion to a second end portion, in a longitudinal side profile each of the first and second end portions extending convexly upward and being separated by an upwardly concave intermediate mid-portion, the first end portion having a curvature selected to support the user's back in juxtaposed contact therewith with an increased disc space height in the anterior side of the cervical, thoracic and lumbar regions relative to their posterior side while the upwardly concave mid-portion engages and supports the sacral and lower lumbar regions of the spine.

14. The physical therapy apparatus as claimed in claim 13, wherein the first end portion extends in a lateral direction from a first support pad side, the first end portion including raised lateral side portions each adjacent the first and second support pad sides, respectively, and a raised central portion being generally aligned with the central axis.

15. The physical therapy apparatus as claimed in claim 13, wherein the second end portion extends in the lateral direction from the first pad side to the second pad side, in lateral profile the second end portion including a recessed central portion generally aligned with the axis, and with a pair of longitudinally elongated recessed side portions, each recessed side portions being located intermediate the recessed central portion and a respective one of the first pad side and the second pad side.

16. The physical therapy apparatus as claimed in claim 13, wherein in side profile, the second end portion has a curvature selected to support the user's neck and shoulders in substantially juxtaposed contact therewith while the first end portion supports the sacral and pelvic regions effecting increased anterior spinal disc space height of the user's lumbar and thoracic sections relative to their posterior side.

17. The physical therapy apparatus as claimed in claim 13, wherein the support pad is symmetrical about the longitudinal central axis.

18. A physical therapy apparatus for effecting vertebral column alignment, re-centering spinal disc cores, decompression of spinal discs and relaxation of soft tissues, the apparatus including: a frame and a body supporting panel mounted on said frame, from a first generally upwardly convex portion to a second generally upwardly convex portion, the body supporting panel extending longitudinally along an axis and including:
an upwardly concave portion intermediate to and connecting said first and second convex portions,
the first convex portion including a generally axially disposed raised members and a pair of recessed portions, each recessed portion disposed on each lateral side of the raised member sized to support the user's shoulder blades therein, in one procedure and the pelvic structure in another procedure,
the concave portion including an inclined surface for supporting the user's lunbar and thoracic vertebrae when the user's spinal column is positioned in juxtaposed contact therewith, the inclined surface extending at an angle selected at about 30° to 50±10° relative to horizontal, and preferably about 45±10° relative to horizontal.
